(19) United States
(12) Patent Application Publication
(10) Pub. No.: US 2006/0141570 A1
(43) Pub. Date: Jun. 29, 2006

(54) Intein-mediated protein purification using in vivo expression of an aggregator protein

(76) Inventors: David W. Wood, Princeton, NJ (US); Mahmoud R. Banki, Princeton, NJ (US); Tillman U. Gerngross, Hanover, NH (US)

Correspondence Address: CONNOLLY BOVE LODGE & HUTZ, LLP, P O BOX 2207, WILMINGTON, DE, 19899, US

(21) Appl. No.: 11/274,254

(22) Filed: Nov. 16, 2005

Related U.S. Application Data
(60) Provisional application No. 60/628,443, filed on Nov. 16, 2004.
(60) Provisional application No. 60/647,339, filed on Jan. 26, 2005.
(60) Provisional application No. 60/661,559, filed on Mar. 14, 2005.

(51) Int. Cl. C12P 21/06; C07H 21/04; C12P 7/62; C12N 9/18; C12N 1/21; C12N 15/74

(52) U.S. Cl. 435/69.1; 435/197; 435/252.33; 435/488; 536/23.2; 435/135

(57) ABSTRACT

Purification of recombinant proteins is performed by expressing in a host cell a fusion protein comprising: (a) a product protein domain, (b) an intein, and (c) at least one aggregator protein domain, wherein the aggregator protein domain comprises a protein that is capable of specific association with granules of polyhydroxyalkanoate (PHA).

### CROSS REFERENCE TO RELATED APPLICATIONS

This application asserts priority to U.S. Provisional Application Nos. 60/628,443 filed Nov. 16, 2004, 60/647,339 filed Jan. 26, 2005, and 60/661,559 filed Mar. 14, 2005, each of which is incorporated herein by reference in its entirety.

### GOVERNMENT LICENSE RIGHTS

The U.S. Government may have certain rights in this invention as provided for by the terms of grant W911 NF-04-1-0056 awarded by the Army Research Office, grant 2000-DT-CX-K001(S-1) awarded by the Department of Justice, grant 60NANB 1 D0064 awarded by the National Institute of Standards and Technology, and grant DAAD 19-00 awarded by the Army Research Office.

### FIELD OF THE INVENTION

The invention is directed generally to methods and compositions for purification of recombinant proteins. More particularly the invention is directed to a method for bioseparation using a fusion protein comprising the desired protein, a self-cleaving intein, and a tag. The fusion protein is associated with a non-soluble cell component through the tag. The non-soluble components are then separated from the soluble components of the cell culture system and optionally washed. The fusion protein is then cleaved by activating the self-cleaving intein. This releases the desired product protein into solution where it can be recovered independent of the intein and tag.

In a preferred method of the invention, the host cell produces the desired protein and the proteins needed to purify it.

### BACKGROUND OF THE INVENTION

Advances in protein expression systems have made possible the production of virtually any oligo peptide or polypeptide product. After expression, however, these products must often be purified for further use. Thus the rapid and economical purification of recombinant proteins represents a persistent challenge in the field of biotechnology. Protein purification typically involves several chromatographic steps, each optimized for each product protein. Each step can be costly and time-consuming, and inevitably decreases the final yield of the product. In the large-scale manufacture of recombinant proteins for industrial and therapeutic use, downstream purification is very costly and can account for up to 80% of the total production cost. The development of simple and reliable methods for protein purification, which can be applied to many products at laboratory to manufacturing scales, is therefore an important goal in bioseparations technology development.

The purification of protein may be obtained by the addition of an affinity tag nucleic acid sequence to a nucleic acid sequence which encodes a target protein. LaVillie et al., Biotechnology 6:501-506 (1995). This process results in the expression of an affinity-tagged target protein that can be purified by exploiting the highly selective binding characteristics of the tag. Once the affinity-tagged target protein is purified, the tag can be enzymatically removed by hydrolysis with an appropriate protease enzyme. Recovery of a native target protein, which is often necessary for many applications, requires the proteolytic removal of the affinity tag. The potential of this technique for use in large scale production is limited in part by complications arising from the addition of protease to the purified fusion protein solution. The protease may cause nonspecific cleavage within the target protein, leading to the destruction of the target protein. A second disadvantage is cost, as protease is expensive. Particularly for industrial applications, protease cost may be a determining factor in selecting a separation system. Also, the addition of protease necessitates an additional purification step for protease removal, which increases costs.

Another method for protein purification involves the creation of a fusion protein in which an intein is inserted between the desired product protein and an affinity binding protein, effectively generating a self-cleaving tag. Discovered in 1990, inteins are naturally occurring internal interruptions in a variety of host proteins. Hirata et al., J. Biol. Chem. 265:6726-6733 (1990); Kane et al., Science 250:651-657 (1990); Perler et al., Nucl. Acids Res. 22:1125-1127 (1994); and Noren et al., Angew. Chem. Int. Ed. 39:450-466 (2000). Inteins are a widely-distributed class of self-splicing protein elements. Protein splicing is a form of posttranslational processing that involves the excision of an intervening protein sequence from a host protein. Concomitantly the flanking polypeptides are joined. The intervening protein sequence is known as an intein, while the flanking sequences are called exteins.

Structural analysis suggests that inteins are generally composed of an endonuclease protein domain and a self-splicing mini-intein domain. The endonuclease domain is not necessary for splicing. Indeed, the endonuclease domain can be deleted to yield a functional splicing mini-intein. One example of a mini-intein is the deletion of the entire endonuclease component from the Mycobacterium tuberculosis recA gene, which reduces the 440 amino acid intein to a functional mini-intein of 168 amino acids.

The genetic elements that encode inteins must be in-frame insertions in a gene with the mature protein product being the same size as the homologs lacking the intein insertion. In addition, the presence of specific splice junctions is necessary. The requisite splice junctions for inteins are serine (Ser, S), threonine (Thr, T) or cysteine (Cys, C) at the intein N-terminus and the dipeptide histadine-asparagine (His-Asn, H—N) or histidine-glutamine (His-Gln, H-Q) at the C-terminus. Ser, Thr, Cys and Asn are necessary residues in the splicing mechanism, and act as nucleophiles to create an N—S or N—O acyl rearrangement, depending on the residue. This forms a linear thioester or ester intermediate. Extein ligation follows, mediated by the highly conserved cysteine, serine or threonine immediately following the intein. Acting as a nucleophile, the sidechain of this residue attacks the ester bond formed in the first step, resulting in transesterification. A branched intermediate is formed. Next, the intein is released when the asparagines at the end of the intein cyclize to form a succinimide. Lastly, an O—N or S—N acyl rearrangement converts the ester linking the exteins to a peptide bond.

Intein function can be modified. For example, a modified intein cleaves instead of splices. Specifically, when an inteins' N-terminal Cys is replaced with an Ala, N-terminal cleaving and splicing is eliminated with C-terminal cleavage observed. Replacing the Asn in the C-terminal with Ala stops C-terminal cleavage and splicing and results in N-terminal cleavage. Other conditions result in cleavages at both the N- and C-terminals, in place of splicing. In the case of C-terminus cleaving, the requirement for a cysteine, serine or threonine immediately following the intein is eliminated.

Thus blocking certain splicing steps permitted the development of self-cleaving affinity tags. Wood and co-workers used the Mycobacterium tuberculosis (Mtu) RecA intein for protein purification with C-terminal cleavage of the target protein. Biotechnol Prog 16(6): 1055-63 (2000). Wood and colleagues also characterized Mtu RecA inteins with the endonuclease domain deleted, creating mini inteins. Furthermore, they were able to create mutated rapid-splicing and cleaving varieties. Characterization showed that the mini-cleaving intein ΔI—CM was very useful for protein purification. Wood et al., Naure Biotechnol. 17(9):889-92 (1999).

Chong and colleagues developed a single-column purification system using the vacuolar ATPase intein subunit of Saccharomyces cerevisiae (Sce VMA intein). Nucleic Acids Res 26(22): 5109-15 (1998). In each case, the intein was inserted in between the affinity binding protein and the product gene. Cells were induced to overexpress precursor protein followed by conventional purification with affinity binding domains. In both cases, the product protein can then be cleaved from the intein affinity tag while on the column, allowing the recovery of the product protein without addition of protease. With the Mtu intein system, the intein cleaving is induced by shifting pH and temperatures. With the Sce intein system, intein cleaving is induced by mass action by the addition of thiol-containing compounds. Additional systems have now been reported that use similar strategies to both systems for inducing intein cleaving. Southworth et al., Biotechniques 27:110-20 (1999).

A remaining practical limitation to the use of self-cleaving affinity tags is the high cost of the affinity resins that are typically used in these separations. Also, the affinity resins often used with inteins have low binding capacity for the tagged fusion proteins, resulting in yield loss.

Applicants have discovered a protein separation system that involves the use of polyhydroxyalkanoates (PHAs). PHAs form granular inclusion bodies in many bacteria and may be intracellular aliphatic carbon storage reserves. The PHA polymer consists of repeating units with the general form —[O—CH(R)(CH₂)ₓCO]ₙ—, the most common of which is polyhydroxybutyrate (PHB) —[O—CH(CH₃)CH₂CO]ₙ. PHB polymer granules have been produced in a wide variety of protein expression systems through simple genetic modification. These systems include many bacterial and yeast systems, including Escherichia coli (Fidler et al., FEMS Microbiol. Rev 9: 231-235 (1992)) and Saccharomyces cerevisiae (Leaf et al., Microbiology 142(pt5): 1169-1180 (1996)), as well as transgenic plant cells (John et al., Proc. Natl. Acad. Sci. 93: 12768-12773 (1996); Hahn et al., Biotechnol. Prog. 15: 1053-1057 (1999)). The macroscopic size and relatively high density of the granules allows them to be easily recovered by a variety of mechanical means following cell lysis.

### SUMMARY OF THE INVENTION

The invention is directed generally to a rapid and highly effective method for preparing substantially purified recombinant protein. The method is highly scaleable and relatively inexpensive. The invention is also directed to fusion proteins, plasmids, cells and compositions useful in the method.

The invention avoids the disadvantages of prior art affinity purification because no separate proteases need be used. Furthermore, the present technology avoids harsh chemical environments. The present invention further eliminates the requirement for conventional affinity tags as well as associated resins and apparatus. The present technology is useful for the expression and extraction of a wide range of proteins. The present invention will permit high quality, low cost preparations of isolated and purified proteins for laboratory and industrial use, such as for purification of industrial enzymes, veterinary products and pharmaceutical products.

In one aspect the invention is directed to a fusion protein comprising a product protein domain, a self-cleaving intein, and at least one aggregator protein domain, wherein the aggregator protein domain comprises a protein that is capable of specific association with granules of polyhydroxyalkanoate (PHA). The intein is located between the product protein domain and the aggregator protein domain. The aggregator protein domain may be one or more phasins that associate with PHA. If it is more than one phasin, the phasins may be linked by an amino acid linker.

In one embodiment, the product protein domain, the intein, and the aggregator protein domain are encoded by a single open reading frame in a nucleotide. In another embodiment, a linker peptide is linked to at least one aggregator protein domain.

The invention also is directed to nucleic acids encoding the fusion proteins of the invention, plasmids comprising the nucleic acids, cells stably transfected with the nucleic acids, and methods of producing the fusion proteins by culturing the cells.

In another embodiment, the invention is directed to methods of purifying a product protein from a recombinant cell culture comprising:
(a) recombinantly producing the fusion protein comprising an aggregator domain comprising at least one phasin and endogenously or through recombinant transfection of phaP genes producing polyhydroxyalkanoates in the same host cell;
(b) allowing the fusion protein and the polyhydroxyalkanoate to leave the host cell either by cell secretion or cell lysis, independently of one another;
(c) allowing the fusion protein to aggregate with the polyhydroxyalkanoate to form a first precipitate;
(d) separating the first precipitate from unprecipitated components of the cell culture medium;
(e) adding water to the first precipitate to form an aqueous precipitate mixture and adjusting one or more conditions of pH, temperature, salt concentration and/or sulfhydryl content of the aqueous precipitate mixture such that the intein self-cleaves from the product protein to form a phasin-intein fusion that remains aggregated with the polyhydroxyalkanoate precipitate and a separated product protein that goes into solution; and
(f) separating the solution of separated product protein from the phasin-intein precipitate to yield a substantially purified protein.

The invention also comprises the protein product isolated by the method of the invention.

### BRIEF DESCRIPTION OF THE FIGURES

FIG. 1 illustrates conventional affinity-based protein purification (A) and protein purification using an intein sequence and a PHB affinity tag (B).

FIG. 2 illustrates the purification of Green Fluorescent Protein expressed in R. eutropha using a PHB affinity based purification method.

FIG. 3 is a vector map of pET21(+)/PPIM. The DNA construct for the phasin-phasin-intein-maltose binding domain is shown. Key restriction enzymes which can be used for cloning are also shown. This is an expression vector under a T7 promoter.

FIG. 4 is a vector map for pET21(+)/PPPIM. The DNA construct for the phasin-phasin-phasin-intein-maltose binding domain is shown. Key restriction enzymes which can be used for cloning are also shown. This is an expression vector under a T7 promoter.

FIG. 5 illustrates scanning electron micrographs of PHB granule formation in E. coli: (A) BLR strain carrying pJM9131, (B) BLR strain carrying a control plasmid, (C) BLR strain carrying pJM9131 plasmid in lactate-supplemented medium, and (D) XL-1-Blue strain carrying pJM9131 plasmid in lactate-supplemental medium.

FIG. 6 illustrates SDS-PAGE results for phasin affinity to PHB.

FIG. 7 illustrates SDS-PAGE showing the purification of maltose-binding protein (denoted as M in FIG. 7).

FIG. 8 illustrates the purification of (a) β-galactosidase (β-gal), (b) chloramphenicol acetyltransferase (CAT), and (c) Nus A protein.

### DETAILED DESCRIPTION OF THE INVENTION

The present invention is envisioned to be used to purify any full size protein, polypeptide or oligo-peptide. As used herein “protein” and “polypeptide” are synonymous. More specifically, the product proteins include, but are not limited to, regulatory factors such as hormones and cytokines; therapeutic polypeptides such as blood products (including coagulation factors), vaccines, and growth hormones; enzymes useful for industrial application such as proteases; remediation enzymes such as organo phosphohydrolases; polynucleotide restriction enzymes; starch hydrolases for mono- and oligo-saccharide manufacture; and antibodies for diagnostic and therapeutic applications. Further, the system can be used in high through-put screening for the parallel purification of large libraries for research purposes. These might include proteomic studies as well as directed evolution and novel enzyme identification studies.

The fusion proteins of the present invention are proteins encoded by multiple in-frame nucleic acid sequences each directed to different protein domains or other copies of the same protein.

In the invention, an intein is used between the product protein domain and the aggregator protein domain as a readily cleavable element that can be used to release the product protein from the fusion protein after purification steps are performed. The inteins used in the present invention are self-cleaving elements in which cleavage can be controlled by pH, temperature, salt concentration, free sulfhydryl concentration and other means that do not involve contact of the intein with an external protease. Intein self-cleavage can be induced by a trigger specific to the intein. Common triggers include addition of a reducing agent such as a thiol and a decrease in pH, for example, from pH 8.5 to 6.0. In one embodiment of the invention, the fusion protein has an intein bound to the product protein at the C-terminus of the intein. In such a fusion protein, intein self-cleavage is desired at the C-terminus, which can be accomplished by change in pH and/or temperature typically. In another embodiment of the invention, the fusion protein has the intein bound to the product protein at the N-terminus of the intein. In this instance, intein self-cleavage is desired at the N-terminus, which may be accomplished by altering the free sulfhydryl concentration.

Preferred are so-called “mini-inteins” in which the endonuclease domain has been deleted, rendering the intein smaller yet still capable of self-cleavage. Examples of such inteins are the pH-sensitive mutant inteins described in Wood et al. Nature Biotechnology 17: 889-892 (1999). Particularly useful is ΔI—CM intein disclosed therein. The ΔI—CM intein is encoded by the sequence found at SEQ ID NO: 1. A key feature of the ΔI—CM mutant is its extreme pH sensitivity, which allows purification of intact precursor followed by rapid C-terminal cleavage. Other useful inteins are found in U.S. Pat. No. 6,933,362 (Belfort et al.). Examples of a useful intein is an intein derived from Mycobacterium tuberculosis (Mtu) recA intein that has only the first 110 amino acids and the last 58 amino acids of that 441-amino acid protein and mutants derived therefrom using methods known in the art. Such an intein is a truncated Mtu recA intein with the endonuclease domain deleted.

Preferred inteins for the present invention display rapid cleavage isolated at either the C-terminal or the N-terminal, more preferably at the C-terminal, and are highly controllable. The cleavage preferably is completed (about 90-95%) in four hours or less at 4° C. or in only minutes at higher temperatures, which allows for easy scaleup. In one embodiment, the inteins used in the invention display a strong dependence on temperature, allowing uncleaved precursor to be expressed in host cells for purification as long as the culture temperature is below the cleavage temperature of the intein. Preferably, the self-cleaving intein yields optimized controllable cleavage rather than splicing. Furthermore, the intein should be as small as possible for this strategy to be attractive for scaleup. Preferred inteins exhibit a 20- to 40-fold increase in activity between pH 8.5 and 6.0. These pH values are relatively mild, decreasing the potential for damage to the product protein due to pH-induced denaturation, and thus allowing the recovery of pure protein with minimal damage. This small pH change also decreases the possibility that the binding domain will lose affinity during cleavage.

Preferably, the intein used allows for self-cleavage that releases the product protein in its native form. An example of such an intein is the C-terminal cleaving ΔI—CM. Other fusion proteins may be used in which self-cleavage of the intein results in modifications to the product protein requiring additional processing to obtain the product protein in native form. For example, in the configuration where the product protein is released by N-terminal cleavage, the cleavage reaction may require the addition of thiol containing compounds that modify the C-terminus of the product protein. Native protein is recovered only after subsequent hydrolysis of the cleavage-inducing reagent. Chong et al., J. Biol. Chem. 272:15587-15590 (1997).

Most preferred inteins are mini-inteins that display rapid, isolated C-terminal cleavage and are pH-sensitive. Such inteins obviate the need for reducing reagents and additional purification steps required for other inteins, such as the N-terminal cleaving inteins discussed supra, and have advantageous size and stability characteristics.

Useful inteins for the present invention include those that have a C-terminal histidine-asparagine. The fusion protein of the invention includes a product protein and an intein, wherein the C-terminal histidine or asparagine or histidine-asparagine of the intein is immediately followed by the second amino acid of the desired product protein. The second amino acid of the desired product protein can be lysine. The presence of the penultimate C-terminal histidine residue may confer pH sensitivity. Thus, it may be advantageous that the C-terminal histidine be present. Preferably the C-terminal asparagine is present for cleavage activity. More particularly, without necessarily wishing to be bound by any one particular theory, it is believed that the mechanism of intein cleavage requires that the final residue of the intein be asparagine (not histidine). The C-terminal histidine referred to herein can be the highly conserved histidine that immediately precedes the final asparagine. If the C-terminal histidine of the intein is immediately followed by the desired product protein and there is no asparagine residue at the final intein residue, then cleavage may not always be possible. The mention herein of a dipeptide at the end of the intein sequence can be interpreted as “Z-asparagine,” to show that the final asparagine residue of the intein is advantageously present for any cleavage, while the histidine residue that precedes it is thought to be responsible for the pH sensitivity of the intein, i.e., “Z” can be histidine. However, “Z” can be any suitable amino acid, such as an amino acid that confers pH sensitivity, e.g., pH sensitivity outside of the range of when “Z” is histidine; for instance, to shift the range of pH sensitivity of the intein.

In the present invention, the aggregator protein domain provides a protein region that is capable of or associating with an insoluble PHA granule to form a complex having low solubility. In this manner, the aggregator protein domain provides a mechanism to separate the fusion protein from the cell lysate or cell culture medium by phase. Chromatography is not required for purification, although it is envisioned that when very high purity is required, the purification method of the present invention may be followed by additional downstream purification steps.

In one embodiment, the aggregator protein domain comprises one or more phasins. In this embodiment of the invention, the phasins are capable of binding to a PHA. The many different PHAs that have been identified to date are primarily linear, head-to-tail polyesters that are composed of 3-hydroxy fatty acid monomers. Preferred PHAs for the present invention are PHB or the copolymer poly(3-hydroxy-butyrate-co-3-hydroxyvalerate) (PHB-co-V), preferably PHB. The method of the invention then involves the presence of a PHA, such as PHB, in the purification system such that the phasin element of the fusion protein can bind to the PHA granules and thereby remove the fusion protein from solution. The fusion protein binds to PHA granules through the phasin domain and is then separated from the cell lysate by centrifugation and separation of the supernatant, by diafiltration using ultrafiltration membranes, by flocculation, gas bubbling, or other methods known to those skilled in the art for separating solid and liquid phases.

The PHA may be produced recombinantly by transfection of the host cell that expresses the fusion protein of the invention with one or more nucleic acids encoding for the proteins involved in cellular biosynthesis of PHA. For example, the genes involved in the biosynthesis of PHB by A. eutrophus have been cloned and expressed in E. coli. Anderson et al., Microbial Reviews 54:450-472, 459 (December 1990). Such a system can be used in the host cells of the present invention. Alternatively, the PHA may be endogenously produced by the host cell. In another embodiment, PHA produced by a different cell or chemically produced is added to the host cell or the cell lysate after fusion protein expression. Sufficient PHA preferably is present to provide for association with all of the phasin in the fusion protein present in the solution.

The PHA granules are structures having low aqueous solubility formed by the aggregation of the polyester product formed from acetyl CoA by the action of Pha A protein (α-ketothiolase, phaA), Pha B protein (a stereo-specific reductase, phaB), and Pha C protein (PHA synthase, phaC).

Linkers may be present in the fusion proteins. Preferred linkers are short, flexible polypeptide domains that allow for the aggregator protein domain or domains to have some conformational flexibility from the product protein domain and thereby encourage aggregation by allowing for the necessary physical conformation to be obtained. The linkers are also found within the aggregator protein domain connecting multiple phasins. Two preferred linkers have the amino acid sequences identified as SEQ ID NOs: 2 and 3. One particular example of a fusion protein is phasin-phasin-phasin-intein-maltose binding domain, in which three phasin protein domains are linked by polypeptide linkers, in which the C terminus of one phasin is linked to the N terminus of an intein and in which the C terminus of the intein is linked to a maltose binding domain. In each case, the various domains of the fusion protein are separated by flexible linkers allowing them to function independently. The exception is that very preferably the C-terminus of the intein is joined directly to the N-terminus of the target protein to allow a native target protein to be recovered following intein cleaving. If the C-terminus of the intein is attached to a linker polypeptide that is then attached to the product protein, additional purification steps may be required after intein cleavage to obtain substantially purified product protein. Although linkers may be used, the invention is not limited to fusion proteins containing linkers. For example, the intein can be contiguous with an aggregator protein domain and the product protein domain.

One advantage of the invention is that it can be used with many different types of host cells. For instance, it is envisioned that the purification system can be used with a prokaryotic cell or a eukaryotic cell. Preferably, the host cell is a bacterial cell, a fungal cell, a mammalian cell, an insect cell, a yeast cell, or a plant cell.

When the fusion protein comprises one or more phasins, then it is preferred that the host cell comprises both the nucleic acid encoding the phasins and also further comprises nucleic acid encoding the three enzymes needed for PHA synthesis: phaA, phaB, and phaC. These enzymes may be endogenously present, or the host cell may be transfected stably or transiently with a plasmid containing the genes for these enzymes. The host cell may be transformed into its chromosomal DNA with the genes encoding phaA, phaB and phaC. In another aspect, the invention comprises a protein expression system comprising a host cell comprising: (a) a nucleic acid plasmid encoding the fusion product of a product protein, an intein and a phasin domain; and (b) a second nucleic acid plasmid encoding a protein useful in the biosynthetic pathway for polyhydroxyalkanoate, preferably for polyhydroxybutyrate.

The plasmid of the invention comprises a nucleotide sequence encoding the fusion protein of the invention. The plasmid can further comprise a promoter sequence, an antibiotic resistance sequence, restriction sites and other elements known in the art that improve the functionality of the plasmid. Preferred is the use of the leaky promoter T7 RNA polymerase such as is described in U.S. Pat. No. 4,952,496.

The invention also relates to a method of purifying a protein comprising isolating the fusion product of the invention from other components of the cell lysate. When the aggregator protein domain comprises a phasin, the fusion protein can be separated from the cell lysate by allowing the fusion protein to associate with a PHA and then isolating the fusion protein/PHA by centrifugation, filtration such as cross-flow-diafiltration, or other means known in the art. In a particular embodiment, the diafiltration uses nanoporous membranes.

In another aspect the invention comprises using the method in a robotic system to purify protein libraries for screening. The purification system of the present invention can be highly automated and thus is suitable for high through-put screening.

### EXAMPLES

### Example 1

### A General Purification Scheme Using PHBs

Introduction

We describe here a protein purification scheme in which the cell produces its own “biological affinity matrix,” thereby eliminating the need for external chromatographic protein purification. This approach is based on the specific interaction of phasin proteins with granules of PHB.

Comparison with Conventional Affinity Separation

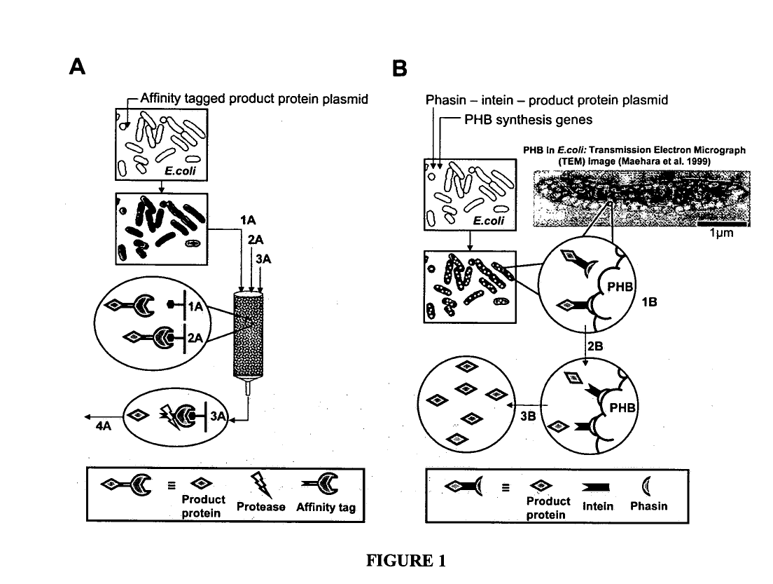

An embodiment of the method of the invention can be compared to conventional means of affinity-based protein purification. See FIG. 1. FIG. 1A illustrates conventional affinity-based protein purification: Cells containing a plasmid for expression of the affinity tag-product protein fusion are induced and harvested. The cell pellet is resuspended, lysed and passed over an affinity resin (1A). The column is then washed to rinse away impurities (2A). The fusion protein is retrieved from the column by addition of excess affinity tag or a displacing substitute. Furthermore, a protease is typically added to cleave off the product protein from the affinity tag (3A). A separation step (4A) salvages the proteases and separates the product protein.

FIG. 1B illustrates the PHB-intein method of affinity-based protein purification: Cells containing two plasmids, one for biosynthesis of PHB granules and another for expression of the phasin-intein tagged product protein, are grown to produce PHB and express the fusion protein. Harvested cells are lysed and centrifuged to separate soluble components (1B). The insoluble PHB granules with the PHB-bound fusion protein fusion are washed and resuspended in a cleavage-inducing buffer for release of the product protein (2B). A final centrifugation separates the PHB granules and associated proteins from the cleaved product protein, leaving only the product protein in the soluble fraction (3B). The cleavage-inducing conditions are tailored to the intein used. Typical conditions are selected from pH shift, a thiol-containing solution, a temperature shift, or combinations of such conditions.

### Example 2

### PHB Purification of GFP

Introduction

By creating in-frame fusions of phasins and green fluorescent protein (GFP) as a model protein, we discovered that GFP can be efficiently sequestered to the surface of PHB granules. In a second step, we generated a phasin-intein-GFP fusion in which the self-cleaving intein was activated by the addition of thiol. This construct allowed for the controlled expression, binding and release of essentially pure GFP in a single separation step.

A protein expression platform based on the Gram-negative bacterium, Ralstonia eutropha is a useful alternative to recombinant protein expression in Escherichia coli.

This example uses the natural ability of R. eutropha to produce PHB, which accumulates as insoluble granules within the cell.

Phasins encoded by the phaP gene (SEQ ID NO:4) accumulate during PHB synthesis, bind to PHB granules and promote further PHB synthesis. Some deletion mutants of phaP form only one large PHB granule. Moreover, up regulating the phaP gene increases the number of PHB granules while reducing their size. Phasins accumulate at high levels in cells that naturally produce PHB, and as much of 5% of total cellular protein can be phasin. Phasins have high affinity for PHB granules, and are the predominant protein present on the granule surface.

The Mxe GyrA intein is a 198 a.a. polypeptide, which has been modified for N-terminal cleavage activity in the presence of thiols. (SEQ ID NO:5) This intein was incorporated into a PhaP-linker-intein-GFP fusion. (SEQ ID NO:6) We were able to show (1) the expression of a PhaP-intein-GFP fusion protein, (2) its sequestration to PHB granules, and (3) the subsequent release of GFP from the PHB granule by treating the cell debris with dithiothreitol (DTT). R. eutropha recombinant strains were generated according to known methods. Srinivasan et al., Appl. Environ. Microbiol. 68: 5925-5932 (2002); Srinivasan et al., Biotech Bioeng. 84: 114-120 (2003).

Methods of Expression in R. eutropha

Plasmid construction. All PCR products were subcloned into pCR 2.1 -TOPO (Invitrogen) and sequence verified. pKNOCK-Cm is a suicide plasmid, conferring chloramphenicol resistance, used for introducing genes into the R. eutropha chromosome. The phaP promoter from pUCPPCm was cloned into pKNOCK-Cm, yielding pGB27. The gfpmut2 gene, a mutant form of a gene that encodes GFP, was PCR amplified from pGY1a+ and cloned into pKNOCK-Cm, yielding plasmid G. A phaP ORF-gfpmut2 ORF translational fusion was constructed by overlap PCR and cloned into pCR 2.1 -TOPO. A peptide linker was introduced between the phaP ORF and gfpmut2 ORF during the overlap PCR. The phaP-gfpmut2 translational fusion was cloned into pKNOCK-Cm and the resulting plasmid designated PG. The Mxe GyrA intein was PCR amplified from pTWIN1 (NEB) and cloned into pCR2.1 TOPO. The intein was cloned into PG, yielding PIG. The exact amino acid sequence encoded by the peptide linker between the phaP ORF and the intein in plasmid PIG is (GGGGS)₃GSGAPM.

R. eutropha strain generation. Methods for introducing plasmids into the R. eutropha chromosome are known. Srinivasan et al., supra. In brief, all pKNOCK-Cm derived plasmids are introduced into E. coli S17 before being transferred into the R. eutropha chromosome by simple biparental mating.

Fluorescence microscopy. To prepare cells for fluorescence microscopy, cells were transferred from LB agar plates into 200 μl of buffer (PBS) and resuspended thoroughly. This cell suspension (10 μl) was transferred to a single well in a 15-well slide pretreated with 1% poly-L-lysine. Microscopy was carried out using a Leica epifluorescence light microscope. An ORCA-ER-CCD camera (Hamamatsu) and OPENLAB software (Improvision) were used for all image acquisition and processing.

Sucrose gradient fractionation. Strains were cultivated in 50 ml of Lee medium (20 g/l glucose, 3 g/l Na₂HPO₄.7H₂O, 1 g/l KH₂PO₄, 2 g/l NH₄Cl, 0.2 g/l MgSO₄.7H₂O, 2.4 ml/l trace element solution, 1 g/l corn steep liquor) to an approximate OD₆₀₀ of 10. The cultures were centrifuged and the cells resuspended in 2 ml of buffer B1 (20 mM Tris, 500 mM NaCl, 1 mM EDTA, pH 8.5). Cells were sonicated in a Fisher Scientific Sonic Dismembrator 550 in ten pulsed cycles (2 seconds ON, 0.5 second OFF, 30 second duration, 5 minute cooling on ice between cycles). 1 ml of the lysate was loaded onto a sucrose density gradient. The sucrose density gradient consists of nine layered 1 ml fractions of buffer B1 containing 0 to 2M sucrose (0.25M increments). The 10 ml solutions were spun at 1,500×g for 3 hours. Ten 1 mL fractions were collected with a syringe needle.

Fluorometry. Fluorescence was measured using the Spetra Max Gemini spectrophotometer (Molecular Devices). Excitation and emission wavelengths of 360 nm and 509 nm respectively, were used.

PHB analysis. The concentration of PHB was quantified by the sulfuric acid-HPLC method of Karr et al. with modifications. York et al., J. Bacteriol. 183, 4217-4226 (2001).

Intein mediated cleavage. 300 μl of the lysate generated from the sonication was centrifuged, the supernatant discarded and the insoluble pellet retained. The pellet was washed three times by resuspension in 1 mL of buffer B1 followed by centrifugation. The pellet was then resuspended in 500 μl of buffer B2 (buffer B1 containing 40 mM DTT). The pellet was incubated overnight by 37° C. After incubation, the solution was centrifuged and supernatant and pellet retained. The pellet was again washed as described above and resuspended in the original in the original volume (500 μl). Samples were subjected to fluorometry and SDS-PAGE (12% Tris HCl polyacrylamide gel (BioRad), stained with SimplyBlue™ SafeStain (Invitrogen)). R. eutropha G, was generated using plasmid pG, which carries a transcriptional fusion between the phaP promoter and the gfpmut2 ORF (phaPp::gfp). Plasmid pG is a suicide plasmid and is integrated at the phaP promoter locus of the R. eutropha chromosome. Since integration occurs within the promoter region, the wild type phaP gene remains intact. R. eutropha PG and R. eutropha PIG were generated using plasmids pPG and pPIG respectively. See Table 1. Plasmid pPG contains an in-frame translational fusion between the phaP ORF and gfpmut2 ORF (phaP::gfp). Plasmid pPIG is isogenic to pPG, with the exception of the in-frame insertion of the Mxe GyrA intein between the two genes (phaP::Mxe GyrA intein::gfp). Plasmids pPG and pPIG do not contain the phaP promoter and the phaP ORF serves as the homologous recombination locus. Therefore in R. eutropha PG and R. eutropha PIG, the wild type phaP gene has been replaced by a translational fusion encoding phaP::gfp and phaP::intein::gfp respectively.

TABLE 1
Plasmids used in Example 2.

| | |
|---|---|
| pKNOCK-Cm | Suicide vector used for introducing plasmids into |
| | R. eutropha chromosome. |
| pCR2.1-TOPO | Commercial high copy number vector used for sub |
| | cloning PCR products.^{a} |
| pUCPPCm | phaPp::oph transcriptional fusion cloned into pUC19. |
| pGB27 | pKNOCK-Cm containing phaP promoter. |
| pGY1a+ | Plasmid vector containing gfpmut2 gene. |
| pTWIN1 | Commercial vector containing Mxe GyrA intein.^{b} |
| pG | phaPp::gfpmut2 transcriptional fusion introduced |
| | into pKNOCK-Cm. Plasmid used to create strain |
| | R. eutropha G. |
| pPG | phaP ORF::gfpmut2 translational fusion introduced |
| | into pKNOCK-Cm. Plasmid used to create strain |
| | R. eutropha PG. |
| pPIG | phaP ORF::Mxe GyrA intein::gfpmut2 translational |
| | fusion in pKNOCK-Cm. Plasmid used to create strain |
| | R. eutropha PIG. |

^{a}Invitrogen
^{b}NEB (Beverly, MA)

Both fluorescence microscopy and sucrose density gradient fractionation of cell lysates were used to examine localization of GFP in R. eutropha strains. Fluorescence microscopy images show that wild type exhibited no autofluorescence and that GFP is evenly distributed throughout the cell in R. eutropha G. Moreover, fluorescent foci are present throughout the cells in R. eutropha PG and R. eutropha PIG, presumably where GFP is localized on the surface of PHB granules.

Sucrose density gradient fractionation of cell lysates was performed to further examine GFP localization. R. eutropha strains were cultivated in Lee medium, a phosphate limited growth medium that induces both PHB formation and transcription of genes under the control of the phaP promoter. Cells were recovered, washed, resuspended in buffer B1 and sonicated. Cell lysates were loaded onto a sucrose gradient (density from 1.02 g/ml to 1.29 g/ml) and equilibrated by centrifugation. PHB granules have a density of approximately 1.20 g/ml and accumulate near the bottom of the sucrose density gradient. In contrast, soluble proteins accumulate in the low density fractions at the top of the sucrose density gradient. A fluorescence spectrophotometer was used to measure the fluorescence of each individual fraction of the sucrose gradient. R. eutropha G showed fluorescence predominantly in the top fractions, consistent with fluorescence micrographs that suggest that GFP is present as a soluble protein in the cytoplasm and not localized to PHB granules.

R. eutropha PG and R. eutropha PIG showed a strong fluorescent signal in a fraction which coincides with the fraction containing PHB. These results strongly suggest that in R. eutropha PG and R. eutropha PIG, the GFP is localized to the PHB granules. Some fluorescent signal also appeared in the upper fractions of the R. eutropha PG and R. eutropha PIG density gradients. Thus PhaP-GFP and PhaP-intein-GFP fusions are localized in vivo to PHB granules. The following demonstrated the release of pure GFP from whole cell debris. Briefly, R. eutropha strains were cultivated in Lee medium, harvested, resuspended in buffer B1 and sonicated. The lysate was centrifuged and the supernatant fraction, containing the soluble protein fraction, was discarded. The pellet was washed in buffer B1. To induce intein cleavage, the pellet was resuspended in buffer B2, and incubated overnight at 37° C. The mixture was then centrifuged and the pellet and supernatant fraction both retained. The pellet was again washed with buffer B1.

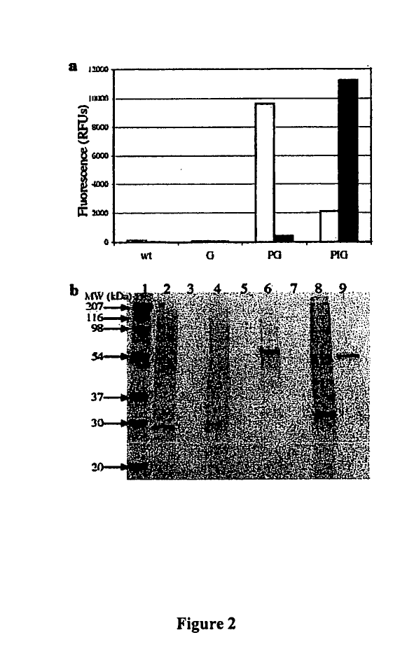

FIG. 2 shows intein mediated cleavage of GFP from whole cell debris. R. eutropha strains were lysed by sonication, the supernatant discarded and the insoluble pellet containing PHB granules retained. Intein mediated cleavage was activated by incubating the washed pellet overnight in buffer B2 at 37° C. After incubation, the pellet and supernatant fractions were isolated. Panel A) shows the results of Fluorometry. Open bars show the fluorescence of the supernatant fractions. Solid bars denote the fluorescence of the resulting pellet fraction. Panel B) shows sodium dodecyl sulfate polyacrylamide gel electrophoresis (SDS PAGE) of fractions. Lane 1 is molecular weight markers. Lanes 2 and 3 are R. eutropha wt pellet and supernatant. Lanes 4 and 5 are R. eutropha G pellet and supernatant. Lanes 6 and 7 are R. eutropha PG pellet and supernatant. Lanes 8 and 9 are R. eutropha PIG pellet and supernatant.

Neither the R. eutropha wt pellet nor the corresponding supernatant showed appreciable fluorescence. Similarly, the pellet and supernatant fractions of R. eutropha G showed no appreciable florescence as expected. As expected, R. eutropha PG showed strong fluorescence on the pellet with no appreciable fluorescence present in the supernatant. In contrast, R. eutropha PIG showed very strong fluorescence in the supernatant fraction, indicating that GFP had been released from the pellet into the supernatant fraction. Although the bulk of the total fluorescence was present in the supernatant, a small amount of fluorescence remained on the PHB granule.

The SDS PAGE showed that the whole cell debris for each strain contains numerous proteins. No protein is visible on the gel for the supernatant fractions of R. eutropha wt, R. eutropha G and R. eutropha PG. The PhaP-intein-GFP fusion protein is expected to be 70 kD in size. If intein mediated cleavage occurs, a protein of 49 kD, corresponding to an intein-GFP (IG) fusion, should be released. FIG. 2B, lane 9, shows that IG was the only protein present in the supernatant fraction. This observation confirms that intein mediated cleavage, activated by thiol addition, released GFP from the granule in the cell debris of R. eutropha PIG.

Thus, the development an integrated protein expression and purification approach, obviates the need for external chromatography. By replacing the wild type phaP gene with a triple translational fusion (phaP ORF, Mxe GyrA intein and gfpmut2), we were able to show that the fusion protein can be localized to the PHB granule and separated from the remaining cytosolic protein fraction by centrifugation. In a subsequent step, we were able to release pure GFP by resuspending whole cell debris (insoluble fraction of cell lysate, containing PHB granules) in a buffer containing DTT.

The single step purification eliminates the need for elaborate and costly protein purification schemes and the undesirable affinity tag (PhaP) remains on the granule. Moreover, adapting the use of inteins eliminates the need for specific endopeptidases, which are routinely used to release recombinant protein from affinity matrixes.

By integrating high-level recombinant protein expression with a simple protein purification step, this system improves upon current technologies for the large-scale production of commodity polypeptides such as enzymes, therapeutic proteins including vaccines, and peptides such as peptide hormones for animal feed.

### Example 3

### Materials and Methods for E. coli-Based Expression

Bacterial Strains, Constructs, and Standard Genetic Manipulations

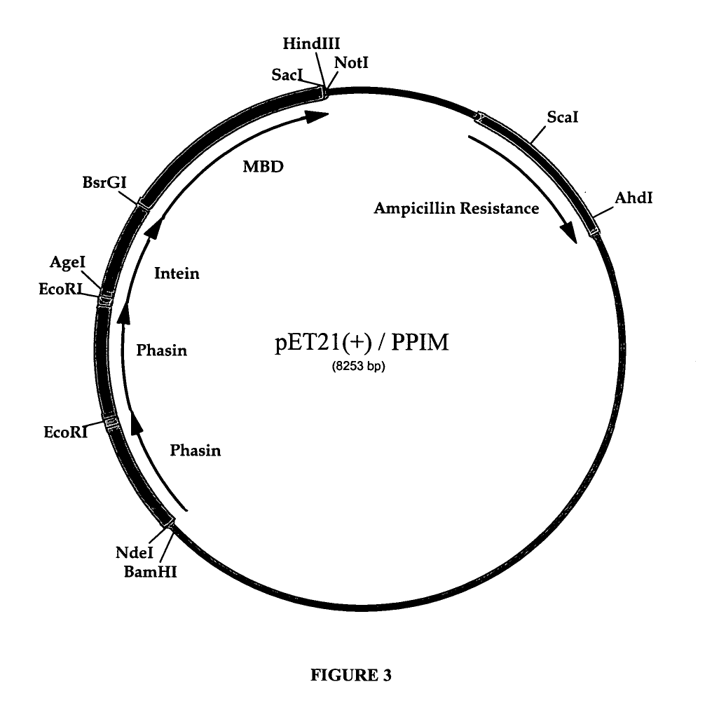

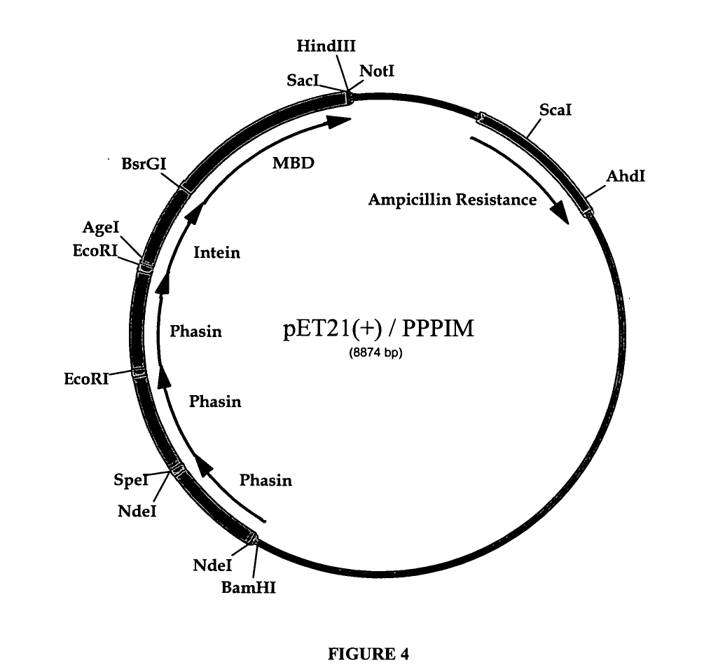

E. coli strains XL1-Blue (recA1 endA1 gyrA96 thi-1 hsdR17 supE44 relA1 lac [F′ proAB lac1^{q}ZΔM15 Tn10(Tet^{R})]) from Stratagene (La Jolla, Calif.), ER2566 (F⁻lamda⁻fhuA2 [Ion] ompT lacZ::T7 gene1 gal sulA11 D(mcrC-mrr)114::IS10 R(mcr-73::miniTn10—TetS)2 R(zgb-210::Tn10)(Tet^{S}) endA1 [dcm]) from NEB (Beverly, Mass.), BL21 (DE3) (F⁻ompT hsdIS_{B} (r_{B}⁻m_{B}⁻) gal dcm(DE3)) and BLR (DE3) (F⁻ ompT hsdS_{B} (r_{B}⁻m_{B}⁻) gal dcm (DE3) Δ(srl-recA)306::Tn10 (Tet^{R})) from Novagen (Madison, Wis.) were used for cloning and expression using standard techniques Sambrook and Russell, Molecular cloning: a laboratory manual, 3rd ed., Cold Spring Harbor Laboratory Press, Cold Spring Harbor, N.Y., 2001. Plasmids pJM9131 (Kan^{R}) containing the phaCAB operon for PHB biosynthesis and phaK (Cam^{R}) containing the phasin phaP gene were kindly provided by Professor Douglas Dennis (Arizona State University, the West Campus) and are described elsewhere. (Kidwell et al., Appl. Environ. Microbiol., 61: 1391-1398 (1995). Plasmid pET-21(+) (Amp^{R}) from Novagen (Madison, Wis.) featuring the T7lac promoter was used for expression and modified by adding a PCR amplified product to include a ribosome binding site and the maltose binding domain (from the pMAL plasmid, New England Biolabs, Beverly, Mass.) between the BamHI and EcoRI sites. After sequence and expression verification for the maltose binding domain (MBD), MBD was replaced by a phasin sequence of SEQ ID NO:4 using NdeI (introduced by the MBD PCR) and EcoRI. The phasin was followed by this linker sequence: AACAATAACAACAACCTCGGGATCGAGGGAAGGATTTCAGAATTC (SEQ ID NO:2). An additional phasin with two flanking NdeI sites was PCR amplified and inserted upstream of the initial phasin. FIG. 3 shows the plasmid vector map for this step. The sequence of the plasmid is identified in SEQ ID NO:7. In the case of the triple phasin constructs PCR amplification was again used to generate a third phasin with two flanking EcoRI sites for insertion downstream of the first phasing as shown in the plasmid vector map at FIG. 4 and at SEQ ID NO:8. PCR amplifications were carried out such that the linker sequence mentioned above followed each inserted phasin in the final construct. The mutated and evolved mini-intein from Mycobacterium tuberculosis (Mtu) recA was digested out of a previous plasmid pMΔ^{†}T-CM (Wood et al., 1999, supra) using EcoRI and BsrGI and was inserted downstream of the phasin sequences (SEQ ID NO:1). The maltose binding domain or other target protein domains, NusA, β-gal, and CAT (SEQ ID NOs:9-12), were PCR amplified flanked by BsrGI and HindIII or NotI and inserted downstream from the intein. The NusA gene came from the pET-43.1 vector available from Novagen (Madison, Wis.). β-gal was PCR amplified from the E. coli chromosome and CAT from the phaK plasmid carrying the Cam^{R} gene.

Media, Expression and PHB Generation

Strains carrying pJM9131 and producing PHB were diluted 100:1 from overnight cultures and grown for 30-hours at 37° C. (unless otherwise noted) in Luria-Bertani medium (1% Bacto tryptone, 0.5% yeast extract, and 1% NaCl) supplemented with 2% sodium lactate and 50 μg/ml kanamycin. In case of double transformants carrying a modified pET-21 vector expressing a fusion protein (such as pET/PPPI:M) the media was additionally supplemented with ampicillin (100 μg/ml). All growth steps were carried out in shake flasks or 5 ml-test tubes in a Labline orbital shaker at 300 rpm. Isopropyl-β-D-thiogalactopyranoside (IPTG) was added for inductions and cultures grown for an additional 4 to 8 hours at 37° C. or 20° C. as indicated, at which point the cells were harvested by centrifugation (5,000 g, 10 min., 4° C.).

Scanning Electron Micrographs

A previously described method (Doi, Microbial polyesters, VCH, New York, N.Y., pp. ix, 156 p., 1990) was modified, to the effect that 1 ml samples grown as described above (in LB+2% lactate for 30 hours) were resuspended in 100 μl lysozyme-containing lysis buffer (10 mM Tris-HCl, 10 mM CaCl₂, 0.5 mg/ml lysozyme) before adding 100 μl of an alkaline-SDS solution (0.4M NaOH, 2% SDS). Four 15-second sonications were carried out on ice allowing the samples to cool between sonications. Samples were dried on carbon tab specimen mounts (Ted Pella) and sputtered with a 2 nm layer of iridium before being examined using a Philips XL30 FEG-SEM under 5 KeV beam.

Purification and SDS-PAGE Analysis

Harvested cell pellets from 1 ml samples were resuspended in 300 μl modified lysis buffer (20 mM Tris, 20 mM Bis, 50 mM NaCl, 1 mM DTT, 2 mM EDTA, 0.25 mg/100 ml lyzozyme, at pH 8.5) and disrupted by ultrasonic disruption at 4° C. Lysed cells were spun in a bench-top centrifuge at 14,000 g for 10-30 minutes at 4° C. Supernatant was then discarded and the cells resuspended in a wash buffer (20 mM Tris, 20 mM Bis, 50 mM NaCl, 1 mM DTT, 2 mM EDTA, at pH 8.5). Resuspended pellet was centrifuged at 14,000 g for 10-30 minutes at 4° C. and the wash discarded. This wash step was repeated as necessary. In the last wash cycle, the pellet was resuspended in a cleavage buffer (20 mM Tris, 20 mM Bis, 50 mM NaCl, 1 mM DTT, 2 mM EDTA, pH 6.5 or pH 6.0) and centrifuged at 14,000 g for 10-30 minutes at 4° C. and the supernatant discarded. This was to ensure homogenous pH throughout the pellet and the tube. The pellet was resuspended again in the cleavage buffer and left to rest at room temperature (18-23° C.) for cleavage. At each time point a total solution fraction was taken and the sample centrifuged at 14,000 g for 10-30 minutes at 4° C. to take a supernatant (soluble) fraction. Samples were resuspended after taking the supernatant time point and left to rest at 20-25° C. for the cleavage to continue to completion. Samples were analyzed by 12% SDS-PAGE followed by staining with Coomassie Brilliant Blue G-250.

Protein Content Quantification & β-gal Activity Assay

Protein concentrations were measured using the Bradford method (Ausubel, Current protocols in molecular biology, John Wiley & Sons, NY, pp. 3v, 1998). β-galactosidase activity assay based on activity with o-Nitrophenyl-β-D-galactopyranoside (ONPG) was measured by the β-gal Activity Assay kit by Stratagene (La Jolla, Calif.).

### Example 4

### Results for E. coli-Based Expression

Production of PHB Granules with Associating Phasin in Expression Strains

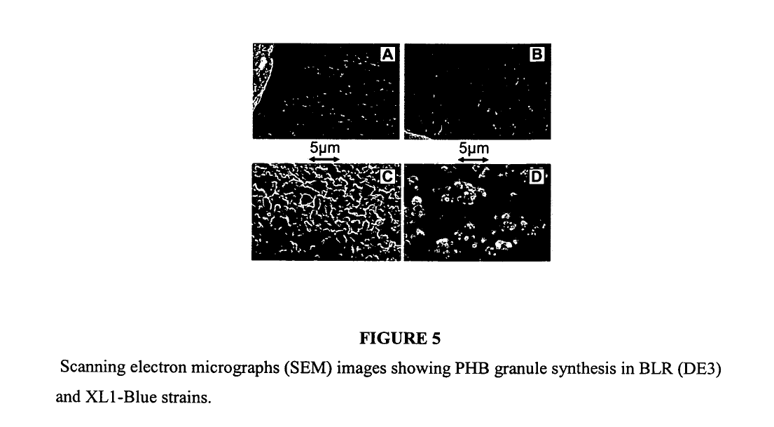

Three enzymes, α-ketothiolase (encoded by the PhaA gene), a stereo-specific reductase (PhaB), and PHA synthase (PhaC), are necessary for transforming metabolic acetyl CoA to PHB and are encoded on plasmid pJM9131. Following published procedures for producing PHB in E. coli XL1-Blue (Pieper-Furst et al., J. Bacteriol. 177, 2513-2523 (1995); Wieczorek et al, J. Bacteriol. 177, 2425-2435 (1995); Maehara et al., FEMS Microbiol. Lett 200, 9-15 (1999), several E. coli laboratory strains were transformed with pJM9131 and grown for 30 hours in LB medium supplemented with 2% sodium lactate as a carbon source for PHB synthesis. Scanning electron microscopy images were prepared of iridium-coated dried cell lysates. FIG. 5 illustrates: (A) BLR strain carrying pJM9131 (PHB biosynthesis plasmid) grown in LB media. (B) BLR strain carrying a control ampicillin resistant plasmid grown in lactate-supplemented LB media. (C) BLR strain carrying pJM9131 grown in lactate-supplemented LB media. (D) XL1-Blue strain carrying pJM9131 grown in lactate-supplemented LB media. The SEM indicated the presence of granules of the expected size (˜100-700 nm) and characteristic shape absent in controls. This result was similar to the SEM images published previously for Alcaligenes eutrophus (Doi 1990, supra), and is in agreement with transmission electron micrographs previously published for PHB production in E. coli XL1-Blue. The E. coli strains XL1-Blue, ER2566, BL21 (DE3), and BLR (DE3) all successfully produced PHB granules when transformed with pJM9131. See, in part, FIG. 5. To assure strong expression of tagged product proteins from the pET-21 vector, BLR (DE3) carrying the T7 RNA polymerase gene was chosen as the host strain for subsequent expression and purification experiments.

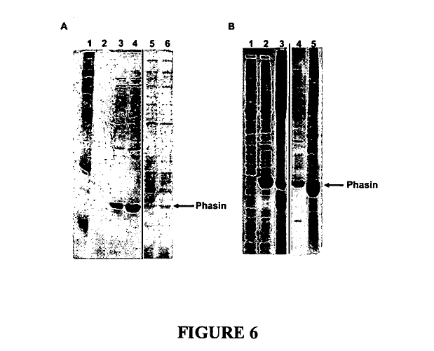

Affinity of the phaP-encoded phasin protein to intracellular PHB granules was examined by expression of the phasin in the presence and absence of co-expressed PHB granules in E. coli cells. The proteins were resolved and identified by SDS-PAGE analysis. See FIG. 6. Panel (A) shows BLR strain carrying phaP gene (plasmid pET/phaP) induced for 0.5 and 2 hours at 37° C. Lane 1 is molecular weight markers. Lane 2 is pre-induction whole-cell lysate. Lanes 3 and 4 are soluble fractions of cell lysates at 0.5 and 2 hour inductions respectively. Lanes 5 and 6 are insoluble fractions corresponding to lanes 3 and 4. The results indicate that phasin expression for 2 hours at 37° C. produced a highly soluble protein in the absence of pJM9131. Panel (B) shows BLR strain carrying the phaP gene (plasmid pET/phaP) and PHB biosynthesis genes (plasmid pJM9131) grown and induced for 8 and 30 hours. Lane 1 is pre-induction whole-cell lysate. Lanes 2 and 3 are soluble fractions after 8 and 30 hours respectively. Lanes 4 and 5 are insoluble fractions corresponding to lanes 2 and 3. Note the displacement of phasin from the soluble fraction (panel B, lane 2) to the insoluble fraction (panel B, lane 5) in the presence of PHB (after 30 hours of growth). Thus, in strains transformed with pJM9131 and grown for 30 hours to produce PHB granules in addition to phasin, the phasin was displaced from the soluble fraction of the lysate to the insoluble pellet. An earlier time point of these double transformants shows that the phasin remains in the soluble fraction prior to PHB production regardless of the presence of pJM9131. This result demonstrates phasin affinity to PHB.

### Example 5

Purification of Maltose Binding Protein

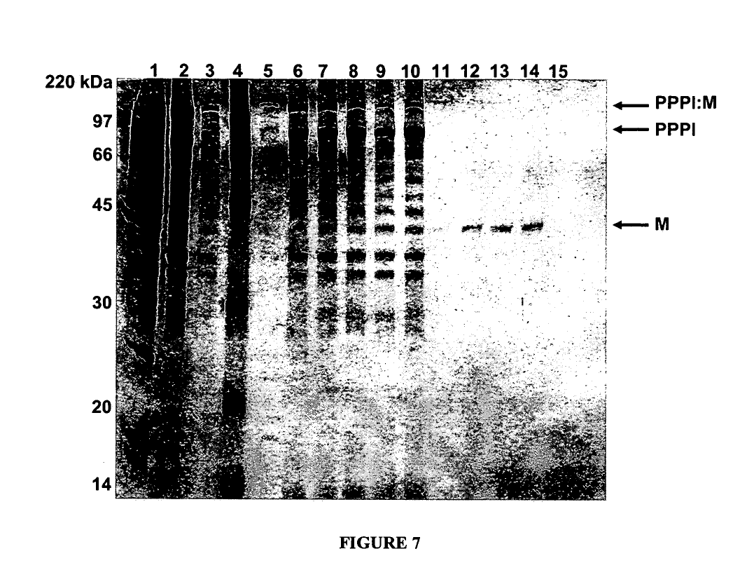

The maltose-binding protein (M or MBP) (SEQ ID NO:9) was prepared as follows. Expression tests indicated that although the phasin alone has high affinity for PHB, fusion proteins of the phasin with the intein and various product proteins had noticeably lower affinity. This led to leakage of the phasin-tagged precursors during the purification procedure, resulting in unacceptable losses in yield. Therefore multiple phasins, separated by flexible linker peptides, were included in the binding tag to enhance fusion affinity to PHB and improve recovery. In particular, three phasins were combined with an engineered mini-intein and the maltose binding domain (MBD) to form PPPI:M (Phasin-Phasin-Phasin-Intein:MBD). A linker peptide joins the phasin domains. The intein is ΔI—CM mini-intein, engineered from the splicing domain of the Mycobacterium tuberculosis (Mtu) recA intein to self-cleave upon application of a pH or temperature shift (Wood et al. 1999, supra). BLR strain was double transformed with pJM9131 and pET/PPPI:M, grown for 24 hours at 37° C. in lactate-supplemented media and then IPTG-induced for an additional 4 hours at the same temperature. In FIG. 7: Lane 1 is the supernatant fraction of the cell lysate. Lane 2 is the insoluble fraction of the cell lysate. Lanes 3 and 5 are decanted wash. Lane 4 is molecular weight markers. Lane 6 is post-wash pellet. Lanes 7-10 are insoluble fractions for the cleavage time course after 1, 3, 20, and 25 hours respectively. Lanes 11-14 are soluble fractions corresponding to lanes 7-10, respectively. Lane 15 is supernatant from lane 14 after addition of maltose resin and centrifugation. The results show C-terminal cleavage of the intein after expression of PPPI:M released the maltose binding protein (M) from the triple-phasin-intein (PPPI) complex. The PPPI:M fusion-protein gene was inserted into the T7 expression vector pET 21(+) to form pET/PPPI:M.

Double transformants carrying PHB biosynthesis genes (pJM9131) and the PPPI:M expression plasmid (pET/PPPI:M) were grown for 30 hours in lactate-supplemented medium to produce PHB granules, at which point overexpression of the PPPI:M fusion protein was induced by IPTG addition. After four more hours of incubation the cells were recovered by centrifugation and lysed by sonication into a pH 8.5 buffer. The intein cleaving reaction is suppressed at this pH, allowing the precursor to be stabilized in an uncleaved form during subsequent granule wash steps. The soluble and insoluble fractions of the resulting cell lysates were separated by centrifugation and analyzed by SDS-PAGE (FIG. 7, lanes 1 and 2). The insoluble pellet, containing the PHB granules and any bound proteins, was washed several times by repeated centrifugation and resuspension in fresh pH 8.5 buffer. The pH was then shifted to 6.0 in the final wash to initiate the intein self-cleavage reaction (FIG. 7, lanes 3 and 5). Unclarified samples (including both soluble and insoluble material) were collected during the cleaving reaction and analyzed for cleavage product formation (FIG. 7, lanes 7-10). Each of these samples was then clarified by centrifugation and the corresponding supernatant was analyzed to detect cleaved soluble product proteins (FIG. 7, lanes 11-14). The results indicate that during incubation over 25 hours at 20° C. the PPPI:M fusion protein cleaves to yield PPPI and M. PPPI was retained in the insoluble phase with the PHB granules, while M (MBP) was released into the soluble fraction. Activity of the purified MBP was subsequently confirmed by its affinity for maltose resin (FIG. 7, lane 15). Similar results were obtained for the double phasin construct of PPI:M. The total MBP yield from this shake-flask experiment was 36.2 mg of MBP per liter of culture (approximately 3.35 mg per gram of dry cell weight). Yields from similar experiments using PPI:M also fell within in the range of 35-40 mg per liter of culture.

### Example 6

Purification of Other Proteins Using a PHB System

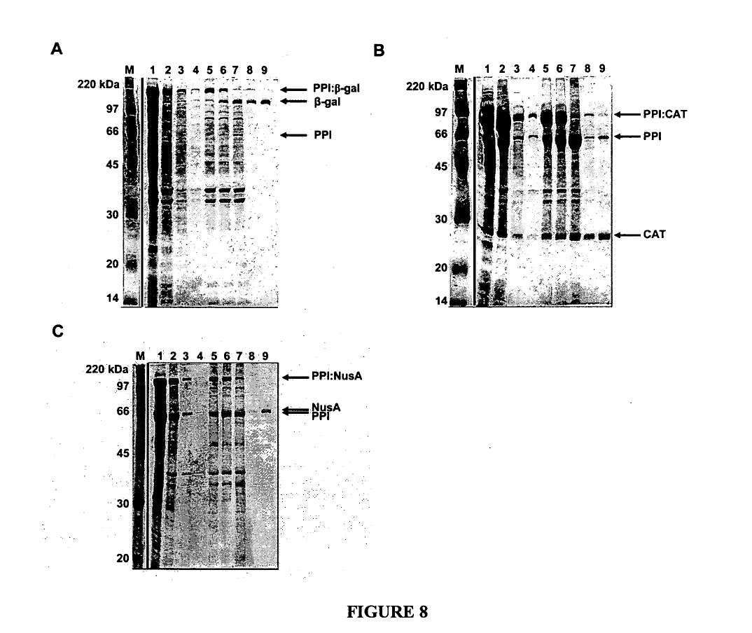

Several additional product proteins were tested using a two-phasin tag. The results are shown in FIG. 8. These proteins included the E. coli μ-galactosidase enzyme (PPI:β-gal; FIG. 8A), the chloramphenicol acetyltransferase (CAT) enzyme (PPI:CAT; FIG. 8B), and the large and highly soluble NusA protein (PPI:NusA; FIG. 8C). The fermentation, induction, and granule washing steps were similar to those described for maltose binding protein. The corresponding lanes in each gel are similar, as follows. Lane M is molecular weight markers. Lane 1 is a supernatant fraction of cell lysate. Lane 2 is an insoluble fraction of cell lysate. Lanes 3 and 4 are decanted wash supernatants. Lane 5 is a post-wash pellet. Lanes 6 and 7 are insoluble fractions for the cleavage time course after 2 and 30 hours respectively. Lanes 8 and 9 are soluble fraction for the cleavage time course after 2 and 30 hours respectively. Samples taken during the cleaving reaction indicated that each product protein was successfully purified at reasonable yield. Lane 9 of each gel in FIG. 8 represents the corresponding purified protein (β-gal, CAT, and NusA) with typical yields of 30 to 40 mg per liter of culture (Table 2). Furthermore, an ONPG assay on the purified β-gal fraction (FIG. 8A, lane 9) verified high yield and activity levels after purification (Table 2).

TABLE 2
Quantification and activity assay of purified proteins

| | Purified protein concentration | | |
|---|---|---|---|
| | | mg/g dry cell | |
| Target Protein | mg/liter culture | weight * | Activity |
| MBP | 36.3 ± 2.2 | 3.35 ± 0.2 | Affinity to Maltose resin |
| β-galactosidase | 39.6 | 3.67 | 91.0 units/mg |
| | | | purified lysate ** |
| CAT | 86.0 *** | 7.96 | N/A |
| NusA | 34.3 ± 2.8 | 3.17 ± 0.26 | N/A |

* Approximate cell pellet weight for 1 ml culture: 27.0 mg. Approximate dry cell weight: 10.8 mg.
** Unit definition: One unit will hydrolyze 1.0 μmole of o-nitrophenyl β-D-galactoside (ONPG) to o-nitrophenol and D-galactose per minute.
*** This protein content includes the impurities (PPI) shown in lane 9 of FIG. 6B.

The purified CAT protein included significant impurities arising from cleaved PPI leaching from the granules into the soluble fraction (FIG. 8B, lane 9). This may arise from the high levels of overexpression of the PPI:CAT fusion relative to the other proteins tested, resulting in saturation of the available PHB granule surface area. This result suggests an upper limit of approximately 5 to 10 milligrams of purified protein per gram dry cell weight for this method. However, as granule size and morphology can be modified by expression levels of phasin protein, significant improvements in yield might be achieved by varying the fusion protein expression levels relative to PHB production.

### Example 7

Isolation of Proteins by Exogenous Addition of Polyhydroxyalkanoic Acid Granules

The method of the invention also encompasses purification of a product protein by binding the fusion protein of the invention to exogenously added PHA (or PHB) granules. In this method a host cell is transfected with a plasmid comprising a nucleic acid encoding a product protein-intein-phasin fusion protein. In the alternative, a plasmid encoding a fusion protein having multiple phasins, each pair optionally linked by an amino acid linker can be used. After host cell growth sufficient to produce the desired amount of product protein, the cells are harvested and lysed. Cell debris is removed by centrifugation or filtration. The clarified supernatant is incubated with PHA granules that have been independently prepared.

PHA granules are prepared from cells producing PHA granules by lysis of the cells followed by centrifugation, filtration, or both. PHA granules can be further purified by mild treatment with detergent and/or density centrifugation. Preferably, host cells that produce PHA granules but that make little or no phasins are used.

After incubation sufficient to permit binding of the fusion proteins to the PHA granules, the complex is collected by centrifugation or filtration. After activation of the intein, and cleavage to release the product protein, the PHA granules, complexed with the remnant fragment of the fusion proteins, are removed. An advantage of this method is that cell debris is effectively removed from the fusion protein.

Advantages of the invention. A strength of this purification method is that the conditions over which it is effective are quite broad, thus providing great flexibility in its implementation. Some optimization will be required for new, uncharacterized products on a case-by-case basis, as is true of any purification method. One of ordinary skill in the art would, however, be able to apply the methods and techniques of the invention to the expression and purification of any desired product protein, based on the extensive guidance provided herein. The presentation of prototypes here aims to exemplify simple means for protein purification that eliminate the high cost and complexity associated with column operation. Although the reduction in cost is somewhat offset by the long induction time and large tags in the fusion proteins, these issues are minor when taken in the context of conventional protein expression and purification. In most of the cases we have shown, the intein cleaving reaction is essentially complete in 4-10 h, making it competitive with any conventional chromatography process. Moreover, the yields we report are reasonable. Furthermore, in one aspect the invention comprises the simple mechanical recovery of precipitated fusion protein by tangential-flow microfiltration or continuous centrifugation.

All references cited herein are incorporated herein by reference in their entirety.

We claim:

1. A fusion protein comprising:
(a) a product protein domain,
(b) a self-cleaving intein, and
(c) at least one aggregator protein domain capable of specific association with granules of polyhydroxyalkanoate (PHA);
wherein the intein is located between the product protein domain and the aggregator protein domain.

2. The fusion protein of claim 1 wherein the intein is ΔI—CM.

3. The fusion protein of claim 1 wherein the at least one aggregator protein domain comprises one or more phasins.

4. The fusion protein of claim 1 wherein the at least one aggregator protein domain comprises one to five phasins that are linked to each other by flexible amino acid linker(s).

5. The fusion protein of claim 3 wherein said one or more phasins are capable of binding to granules of polyhydroxybutyrate.

6. The fusion protein of claim 1 in which the at least one aggregator protein domain is covalently attached to the intein by a flexible amino acid linker.

7. A nucleic acid encoding the fusion protein of claim 1.

8. The nucleic acid of claim 7 wherein the product protein domain, the intein, and the aggregator protein domain form a single open reading frame.

9. A plasmid comprising the nucleic acid of claim 7.

10. A cell stably transfected with the nucleic acid of claim 7.

11. A nucleic acid encoding the fusion protein of claim 3.

12. A plasmid comprising the nucleic acid of claim 11.

13. A cell stably transfected with the nucleic acid of claim 11.

14. The cell of claim 13 that is further stably transfected with nucleic acid encoding phaA, phaB, and phaC.

15. The cell of claim 13 that endogenously produces phA, phaB, and phaC.

16. The cell of claim 15 wherein said cell is a strain from E. coli.

17. A host cell comprising:
(a) a first plasmid encoding the fusion protein of claim 3; and
(b) a second plasmid encoding at least one protein involved in the biosynthesis of a polyhydroxyalkanoate.

18. A method of expressing a fusion protein comprising culturing the cell of claim 10.

19. A method of expressing a fusion protein comprising culturing the cell of claim 13.

20. A method of purifying a product protein from a recombinant cell culture medium comprising:
(a) recombinantly producing the fusion protein of claim 3 and endogenously or through recombinant transfection of phaP genes producing polyhydroxyalkanoates in the same host cell;
(b) allowing the fusion protein and the polyhydroxyalkanoate to leave the host cell either by cell secretion or cell lysis, independently of one another;
(c) allowing the fusion protein to aggregate with the polyhydroxyalkanoate to form a first precipitate;
(d) separating the first precipitate from unprecipitated components of the cell culture medium;
(e) adding water to the first precipitate to form an aqueous precipitate mixture and adjusting one or more conditions of pH, temperature, salt concentration and/or sulfhydryl content of the aqueous precipitate mixture such that the intein self-cleaves from the product protein to form a phasin-intein fusion that remains aggregated with the polyhydroxyalkanoate precipitate and a separated product protein that goes into solution; and
(f) separating the solution of separated product protein from the phasin-intein precipitate to yield a substantially purified protein.

21. The method of claim 20 wherein the first precipitate is separated from the unprecipitated components of the cell culture medium by centrifugation, filtration, flocculation or by settling.

22. The method of claim 20 wherein the at least one aggregator protein domain comprises one to five phasins that are linked to each other by flexible amino acid linkers.

23. The method of claim 20 wherein the polyhydroxyalkanoate is polyhydroxybutyrate.

24. The method of claim 20 wherein the fusion protein and the polyhydroxyalkanoate leave the host cell as a result of cell lysis.

25. The method of claim 20 wherein the intein is ΔI—CM.

26. The method of claim 25 wherein the temperature of the second suspension is adjusted to 18-22° C. and the suspension is incubated such that the intein self-cleaves from the product protein.

27. The method of claim 20 wherein the first precipitate is washed prior to allowing the intein to self-cleave.


SEQUENCE LISTING (12 sequences)

SEQ ID NO: 1 - DNA, 504 bp, Artificial Sequence
  delta-I-CM intein mutant
gccctcgcagagggcactcggatcttcgatccggtcaccggtacaacgcatcgcatcgaggatgttgtcggtgggcgcaagcctattcatgtcgtggctgctgccaaggacggaacgctgcatgcgcggcccgtggtgtcctggttcgaccagggaacgcgggatgtgatcgggttgcggatcgccggtggcgccatcctgtgggcgacacccgatcacaaggtgctgacagagtacggctggcgtgccgccggggaactccgcaagggagacagggtggcgcaaccgcgacgcttcgatggattcggtgacagtgcgccgattccggcgcgcgtgcaggcgctcgcggatgccctggatgacaaattcctgcacgacatgctggcggaagaactccgctattccgtgatccgagaagtgctgccaacgcggcgggcacgaacgttcggcctcgaggtcgaggaactgcacaccctcgtcgccgaaggggttgttgtacacaac

SEQ ID NO: 2 - DNA, 45 bp, Artificial Sequence
  Phasin Linker1 DNA sequence
aacaataacaacaacctcgggatcgagggaaggatttcagaattc

SEQ ID NO: 3 - DNA, 45 bp, Artificial Sequence
  Phasin-linker2 DNA Sequence
aacaataacaacaacctcgggatcgagggaaggattactagtcat

SEQ ID NO: 4 - DNA, 576 bp, Artificial Sequence
  Phasin DNA Sequence
atgatcctcaccccggaacaagttgcagcagcgcaaaaggccaacctcgaaacgctgttcggcctgaccaccaaggcgtttgaaggcgtcgaaaagctcgtcgagctgaacctgcaggtcgtcaagacttcgttcgcagaaggcgttgacaacgccaagaaggcgctgtcggccaaggacgcacaggaactgctggccatccaggccgcagccgtgcagccggttgccgaaaagaccctggcctacacccgccacctgtatgaaatcgcttcggaaacccagagcgagttcaccaaggtagccgaggctcaactggccgaaggctcgaagaacgtgcaagcgctggtcgagaacctcgccaagaacgccccggccggttcggaatcgaccgtggccatcgtgaagtcggcgatctccgctgccaacaacgcctacgagtcggtgcagaaggcgaccaagcaagcggtcgaaatcgctgaaaccaacttccaggctgcggctacggctgccaccaaggctgcccagcaagccagcgccacggcccgtacggccacggcaaagaagacgacggctgcc

SEQ ID NO: 5 - protein, 198 aa, Artificial Sequence
  Mxe GyrA intein obtain from NEB pTWIN1 vector
CITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHA

SEQ ID NO: 6 - protein, 654 aa, Artificial Sequence
  Sequence of PhaP-Polylinker-intein-GFP fusion protein
MILTPEQVAAAQKANLETLFGLTTKAFEGVEKLVELNLQVVKTSFAEGVDNAKKALSAKDAQELLAIQAAAVQPVAEKTLAYTRHLYEIASETQSEFTKVAEAQLAEGSKNVQALVENLAKNAPAGSESTVAIVKSAISAANNAYESVQKATKQAVEIAETNFQAAATAATKAAQQASATARTATAKKTTAAGGGGSGGGGSGGGGSGSGAPMCITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHASRGSIMSKGEELFTGVVPILVELDGDVNGHKFSVSGEGEGDATYGKLTLKFICTTGKLPVPWPTLVTTFAYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 7 - DNA, 8253 bp, Artificial Sequence
  pET21(+)/PPIM plasmid vector DNA sequence
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctaggatccaataattttgtttaactttaagaaggagatatacatatgatcctcaccccggaacaagttgcagcagcgcaaaaggccaacctcgaaacgctgttcggcctgaccaccaaggcgtttgaaggcgtcgaaaagctcgtcgagctgaacctgcaggtcgtcaagacttcgttcgcagaaggcgttgacaacgccaagaaggcgctgtcggccaaggacgcacaggaactgctggccatccaggccgcagccgtgcagccggttgccgaaaagaccctggcctacacccgccacctgtatgaaatcgcttcggaaacccagagcgagttcaccaaggtagccgaggctcaactggccgaaggctcgaagaacgtgcaagcgctggtcgagaacctcgccaagaacgccccggccggttcggaatcgaccgtggccatcgtgaagtcggcgatctccgctgccaacaacgcctacgagtcggtgcagaaggcgaccaagcaagcggtcgaaatcgctgaaaccaacttccaggctgcggctacggctgccaccaaggctgcccagcaagccagcgccacggcccgtacggccacggcaaagaagacgacggctgccaacaataacaacaacctcgggatcgagggaaggatttcagaattcatcctcaccccggaacaagttgcagcagcgcaaaaggccaacctcgaaacgctgttcggcctgaccaccaaggcgtttgaaggcgtcgaaaagctcgtcgagctgaacctgcaggtcgtcaagacttcgttcgcagaaggcgttgacaacgccaagaaggcgctgtcggccaaggacgcacaggaactgctggccatccaggccgcagccgtgcagccggttgccgaaaagaccctggcctacacccgccacctgtatgaaatcgcttcggaaacccagagcgagttcaccaaggtagccgaggctcaactggccgaaggctcgaagaacgtgcaagcgctggtcgagaacctcgccaagaacgccccggccggttcggaatcgaccgtggccatcgtgaagtcggcgatctccgctgccaacaacgcctacgagtcggtgcagaaggcgaccaagcaagcggtcgaaatcgctgaaaccaacttccaggctgcggctacggctgccaccaaggctgcccagcaagccagcgccacggcccgtacggccacggcaaagaagacgacggctgccaacaataacaacaacctcgggatcgagggaaggatttcagaattcgccctcgcagagggcactcggatcttcgatccggtcaccggtacaacgcatcgcatcgaggatgttgtcggtgggcgcaagcctattcatgtcgtggctgctgccaaggacggaacgctgcatgcgcggcccgtggtgtcctggttcgaccagggaacgcgggatgtgatcgggttgcggatcgccggtggcgccatcctgtgggcgacacccgatcacaaggtgctgacagagtacggctggcgtgccgccggggaactccgcaagggagacagggtggcgcaaccgcgacgcttcgatggattcggtgacagtgcgccgattccggcgcgcgtgcaggcgctcgcggatgccctggatgacaaattcctgcacgacatgctggcggaagaactccgctattccgtgatccgagaagtgctgccaacgcggcgggcacgaacgttcggcctcgaggtcgaggaactgcacaccctcgtcgccgaaggggttgttgtacacaacatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgtgactgcaggcaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 8 - DNA, 8874 bp, Artificial Sequence
  pET21(+) PPPIM plasmid expression vector DNA sequence
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctaggatccaataattttgtttaactttaagaaggagatatacatatgatcctcaccccggaacaagttgcagcagcgcaaaaggccaacctcgaaacgctgttcggcctgaccaccaaggcgtttgaaggcgtcgaaaagctcgtcgagctgaacctgcaggtcgtcaagacttcgttcgcagaaggcgttgacaacgccaagaaggcgctgtcggccaaggacgcacaggaactgctggccatccaggccgcagccgtgcagccggttgccgaaaagaccctggcctacacccgccacctgtatgaaatcgcttcggaaacccagagcgagttcaccaaggtagccgaggctcaactggccgaaggctcgaagaacgtgcaagcgctggtcgagaacctcgccaagaacgccccggccggttcggaatcgaccgtggccatcgtgaagtcggcgatctccgctgccaacaacgcctacgagtcggtgcagaaggcgaccaagcaagcggtcgaaatcgctgaaaccaacttccaggctgcggctacggctgccaccaaggctgcccagcaagccagcgccacggcccgtacggccacggcaaagaagacgacggctgccaacaataacaacaacctcgggatcgagggaaggattactagtcatatgatcctcaccccggaacaagttgcagcagcgcaaaaggccaacctcgaaacgctgttcggcctgaccaccaaggcgtttgaaggcgtcgaaaagctcgtcgagctgaacctgcaggtcgtcaagacttcgttcgcagaaggcgttgacaacgccaagaaggcgctgtcggccaaggacgcacaggaactgctggccatccaggccgcagccgtgcagccggttgccgaaaagaccctggcctacacccgccacctgtatgaaatcgcttcggaaacccagagcgagttcaccaaggtagccgaggctcaactggccgaaggctcgaagaacgtgcaagcgctggtcgagaacctcgccaagaacgccccggccggttcggaatcgaccgtggccatcgtgaagtcggcgatctccgctgccaacaacgcctacgagtcggtgcagaaggcgaccaagcaagcggtcgaaatcgctgaaaccaacttccaggctgcggctacggctgccaccaaggctgcccagcaagccagcgccacggcccgtacggccacggcaaagaagacgacggctgccaacaataacaacaacctcgggatcgagggaaggatttcagaattcatcctcaccccggaacaagttgcagcagcgcaaaaggccaacctcgaaacgctgttcggcctgaccaccaaggcgtttgaaggcgtcgaaaagctcgtcgagctgaacctgcaggtcgtcaagacttcgttcgcagaaggcgttgacaacgccaagaaggcgctgtcggccaaggacgcacaggaactgctggccatccaggccgcagccgtgcagccggttgccgaaaagaccctggcctacacccgccacctgtatgaaatcgcttcggaaacccagagcgagttcaccaaggtagccgaggctcaactggccgaaggctcgaagaacgtgcaagcgctggtcgagaacctcgccaagaacgccccggccggttcggaatcgaccgtggccatcgtgaagtcggcgatctccgctgccaacaacgcctacgagtcggtgcagaaggcgaccaagcaagcggtcgaaatcgctgaaaccaacttccaggctgcggctacggctgccaccaaggctgcccagcaagccagcgccacggcccgtacggccacggcaaagaagacgacggctgccaacaataacaacaacctcgggatcgagggaaggatttcagaattcgccctcgcagagggcactcggatcttcgatccggtcaccggtacaacgcatcgcatcgaggatgttgtcggtgggcgcaagcctattcatgtcgtggctgctgccaaggacggaacgctgcatgcgcggcccgtggtgtcctggttcgaccagggaacgcgggatgtgatcgggttgcggatcgccggtggcgccatcctgtgggcgacacccgatcacaaggtgctgacagagtacggctggcgtgccgccggggaactccgcaagggagacagggtggcgcaaccgcgacgcttcgatggattcggtgacagtgcgccgattccggcgcgcgtgcaggcgctcgcggatgccctggatgacaaattcctgcacgacatgctggcggaagaactccgctattccgtgatccgagaagtgctgccaacgcggcgggcacgaacgttcggcctcgaggtcgaggaactgcacaccctcgtcgccgaaggggttgttgtacacaacatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgtgactgcaggcaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 9 - DNA, 1116 bp, Unknown
  Coding Sequence for a Maltose Binding Protein
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgtga

SEQ ID NO: 10 - DNA, 1488 bp, Unknown
  Coding Sequence for NusA
atgaacaaagaaattttggctgtagttgaagccgtatccaatgaaaaggcgctacctcgcgagaagattttcgaagcattggaaagcgcgctggcgacagcaacaaagaaaaaatatgaacaagagatcgacgtccgcgtacagatcgatcgcaaaagcggtgattttgacactttccgtcgctggttagttgttgatgaagtcacccagccgaccaaggaaatcacccttgaagccgcacgttatgaagatgaaagcctgaacctgggcgattacgttgaagatcagattgagtctgttacctttgaccgtatcactacccagacggcaaaacaggttatcgtgcagaaagtgcgtgaagccgaacgtgcgatggtggttgatcagttccgtgaacacgaaggtgaaatcatcaccggcgtggtgaaaaaagtaaaccgcgacaacatctctctggatctgggcaacaacgctgaagccgtgatcctgcgcgaagatatgctgccgcgtgaaaacttccgccctggcgaccgcgttcgtggcgtgctctattccgttcgcccggaagcgcgtggcgcgcaactgttcgtcactcgttccaagccggaaatgctgatcgaactgttccgtattgaagtgccagaaatcggcgaagaagtgattgaaattaaagcagcggctcgcgatccgggttctcgtgcgaaaatcgcggtgaaaaccaacgataaacgtatcgatccggtaggtgcttgcgtaggtatgcgtggcgcgcgtgttcaggcggtgtctactgaactgggtggcgagcgtatcgatatcgtcctgtgggatgataacccggcgcagttcgtgattaacgcaatggcaccggcagacgttgcttctatcgtggtggatgaagataaacacaccatggacatcgccgttgaagccggtaatctggcgcaggcgattggccgtaacggtcagaacgtgcgtctggcttcgcaactgagcggttgggaactcaacgtgatgaccgttgacgacctgcaagctaagcatcaggcggaagcgcacgcagcgatcgacaccttcaccaaatatctcgacatcgacgaagacttcgcgactgttctggtagaagaaggcttctcgacgctggaagaattggcctatgtgccgatgaaagagctgttggaaatcgaaggccttgatgagccgaccgttgaagcactgcgcgagcgtgctaaaaatgcactggccaccattgcacaggcccaggaagaaagcctcggtgataacaaaccggctgacgatctgctgaaccttgaaggggtagatcgtgatttggcattcaaactggccgcccgtggcgtttgtacgctggaagatctcgccgaacagggcattgatgatctggctgatatcgaagggttgaccgacgaaaaagccggagcactgattatggctgcccgtaatatttgctggttcggtgacgaagcgtga

SEQ ID NO: 11 - DNA, 3075 bp, Unknown
  Coding Sequence for a beta-galactosidase
atgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 12 - DNA, 660 bp, Unknown
  Coding Sequence for a Chloroamphenicol acetyl transferase
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaa